(19) United States
(12) Patent Application Publication
(10) Pub. No.: US 2016/0270635 A1
(43) Pub. Date: Sep. 22, 2016

(54) ENDOSCOPE

(71) Applicant: FUJIFILM Corporation, Tokyo (JP)

(72) Inventors: Toshizumi TANAKA, Kanagawa (JP); Tomohiro OHKI, Kanagawa (JP); Teruyuki EMURA, Kanagawa (JP); Sunao HACHISUKA, Kanagawa (JP)

(73) Assignee: FUJIFILM Corporation, Tokyo (JP)

(21) Appl. No.: 15/073,661

(22) Filed: Mar. 18, 2016

(30) Foreign Application Priority Data
Mar. 20, 2015 (JP) 2015-058347

(51) Int. Cl. A61B 1/018; A61B 1/00

(52) U.S. Cl. CPC A61B 1/018; A61B 1/00137; A61B 1/00121

(57) ABSTRACT

According to an endoscope according to an aspect of the present invention, in a cap, an opening window which opens an opening portion on an upper surface side of an elevator housing slit n a state attached to a distal end portion body and a partition wall portion which closes an opening portion on a lower surface side are formed, and in a state in which the cap is attached to the distal end portion body, reclining of an elevator is regulated by the partition wall portion. Moreover, an opening portion from the upper surface to the lower surface through a front surface of the distal end portion body is extended and open, and thus, an exposed range of the elevator is wide, and the elevator can be cleaned rapidly and easily.

### CROSS-REFERENCE TO RELATED APPLICATIONS

The present application claims priority under 35 U.S.C. §119 to Japanese Patent Application No. 2015-058347, filed on Mar. 20, 2015. Each of the above application(s) is hereby expressly incorporated by reference, in their entirety, into the present application.

### BACKGROUND OF THE INVENTION

1. Field of the Invention

The present invention relates to an endoscope and particularly to a structure of a distal end portion of an endoscope provided with an elevator (forceps elevator) for a treatment tool and an elevating mechanism (elevator erecting mechanism) for the elevator.

2. Description of the Related Art

Regarding an endoscope, various treatment tools are inserted into a treatment-tool entry port provided on an operation portion and they are led out of the treatment-tool exit port opened in the distal end portion and used for treatment. The treatment tools such as a guide wire, a contrast medium tube and the like are used for a duodenoscopy, puncture needles for an ultrasonic endoscope, and forceps, snares and the like for a direct-viewing endoscope or a side-viewing endoscope, for example. These treatment tools need to change a derivation direction at the distal end portion in order to treat a desired position in a subject, and thus, a treatment-tool elevating mechanism (forceps elevator, hereinafter referred to as “elevator”) is provided on the distal end portion.

As such the treatment-tool elevating mechanisms, a mechanism in which a wire is attached to the elevator and extended to a proximal end side of the endoscope, is known. In the mechanism, the elevator is rotated around a rotating shaft by pushing and pulling operation of the wire with an operation lever provided on the operation portion so as to change a position of the elevator between an erecting position and a reclining position. Moreover, a mechanism (lever type) in which the rotating shaft of the elevator is coupled with a housed lever through a partition wall, and the wire is attached to the lever is also known. In the mechanism, the elevator is rotated around the rotating shaft by means of the pushing and pulling operation of the wire with the operation lever provided on the operation portion so as to change the position of the elevator between the erecting position and the reclining position.

The distal end portion provided with such a treatment-tool elevating mechanism has a complicated shape and structure and thus, improvement of cleaning performances such as wraparound of a disinfectant, insertion of a washing brush (reachability of a tip end of the brush) or drainage and ease of a cleaning work are in demand.

Conventionally, an endoscope having a detachable cap on the distal end portion is known (see Japanese Patent Laid-Open No. 2004-141315, for example). In this type of endoscope, the cap is removed after treatment, and then the distal end portion is cleaned.

### SUMMARY OF THE INVENTION

However, with the conventional endoscope as described in Japanese Patent Laid-Open No. 2004-141315, the distal end portion itself is originally small-sized and moreover, since the elevator is housed in an elevator housing groove (a slit which houses the elevator), an exposed portion is small. In addition, since a gap between the elevator and the elevator housing groove is narrow, cleaning using a brush or the like takes time.

The present invention was made in view of such circumstances and aims to provide an endoscope whose distal end portion can be cleaned rapidly and easily.

In order to achieve the aforementioned object, an endoscope according to a first aspect of the present invention includes: an insertion portion which includes a distal end and a proximal end; an operation portion which is provided on a proximal end side of the insertion portion and includes an operating member; a distal end portion body which is provided on a distal end side of the insertion portion, and has a front surface, an upper surface, and a lower surface; an elevator which has a rotation center and is configured to be rotatable with respect to the distal end portion body; an elevator erecting mechanism configured to rotate the elevator around the rotation center; an operation wire which includes a proximal-end-side coupling portion coupled with the operating member and a distal-end-side coupling portion coupled with the elevator erecting mechanism, the operation wire configured to rotate the elevator by being pushed or pulled by an operation of the operating member, and displace the elevator between an erecting position and a reclining position; an elevator housing slit which is provided on the distal end portion body and forms a space portion housing the elevator, the elevator housing slit being extended and opened from the upper surface to the lower surface through the front surface of the distal end portion body; and a cap detachably provided on the distal end portion body, the cap including an opening window which opens the opening portion on a side of the upper surface of the elevator housing slit and a partition wall portion which closes the opening portion on a side of the lower surface in a state in which the cap is attached to the distal end portion body.

According to the first aspect of the present invention, since the cap is detachably attached to the distal end portion body, by removing the cap, the elevator is exposed from the opening of the elevator housing slit. Further, since the elevator housing slit is extended from the upper surface to the lower surface of the distal end portion body and opened, an exposed range of the elevator is large, and the distal end portion can be cleaned rapidly and easily. In the first aspect, the endoscope may be configured such that the rotating shaft is fixed to the elevator and the rotating shaft is rotated integrally with the elevator. Alternately, the endoscope may be configured such that the rotating shaft is fixed to the distal end portion body and the elevator is rotated around the fixed rotating shaft. Here, in the present invention, the term “rotation center” of the elevator refers to a virtual rotation center line of the elevator.

An endoscope according to a second aspect is so constituted that, in the first aspect, the elevator has: a forceps action region which has a first width and is configured to receive forceps; a rotation center region which has a second width narrower than the first width and includes the rotation center of the elevator; and an elevator width transition region which is disposed between the forceps action region and the rotation center region, and whose width changes from the second width to the first width, and the elevator housing slit has: a distal-end-side region which has a third width wider than the first width and is configured to house the forceps action region; a proximal-end-side region which has a fourth width narrower than the third width and wider than the second width, and is configured to house the rotation center region; and a forceps housing slit width transition region which is disposed between the distal-end-side region and the proximal-end-side region, and whose width changes from the fourth width to the third width, wherein a distance from the rotation center to the forceps housing slit width transition region is shorter than a distance from the rotation center to the elevator width transition region. The second aspect defines a shape that houses the elevator in the elevator housing slit.

An endoscope according to a third aspect is so constituted that, in the first or second aspect, the elevator erecting mechanism has an elevator erecting lever coupled with the elevator, the distal-end-side coupling portion of the operation wire is coupled with the elevator erecting lever, and the operation wire is pushed or pulled by an operation of the operating member so as to rotate the elevator through the elevator erecting lever and displace the elevator.

The third aspect illustrates an aspect of the elevator erecting mechanism.

An endoscope according to a fourth aspect is so constituted that, in the third aspect, the elevator erecting lever is coupled with the elevator in an unrotatable manner, and the elevator erecting lever is configured to be inclined larger than the elevator toward the proximal end side with respect to the rotation center when the elevator is erected, and a side surface of the elevator is exposed from an opening portion on a side of the front surface of the elevator housing slit or the opening portion on the side of the lower surface of the elevator housing slit, before the elevator erecting lever abuts on the distal end portion body and further rotation of the elevator erecting lever and the elevator is restricted, when the erecting of the elevator is cancelled. By setting such erecting relation between the elevator erecting lever and the elevator, a rotating space of the elevator erecting lever is reduced, and the distal end portion of the endoscope can be downsized.

An endoscope according to a fifth aspect is so constituted that, in one of the first to fourth aspects, the distal end portion body has a pair of side wall portions facing each other having the elevator housing slit therebetween, and at least one side wall portion of the pair of side wall portions has a notch portion which is formed by notching a corner portion on the side of the lower surface, the corner portion being on the side of the front surface, and the notch portion is configured to allow the side surface of the elevator to be exposed when the elevator is displaced to the reclining position. In the fifth aspect, when the elevator is displaced to the reclining position, the notch portion allows the side surface of the elevator to be exposed and thus, the distal end portion can be cleaned more rapidly and easily.

An endoscope according to a sixth aspect is so constituted that, in one of the first to fourth aspects, the distal end portion body has a pair of side wall portions facing each other having the elevator housing slit therebetween, and at least one side wall portion of the pair of side wall portions has a first thick portion which is provided on the side of the upper surface rather than the lower surface and has a first thickness, and a second thick portion which is provided between the first thick portion and the lower surface and has a second thickness smaller than the first thickness. In the sixth aspect, at least one side wall portion of the pair of the side wall portions has the second thick portion, and thus, a gap corresponding to a difference between the first thickness and the second thickness is generated between the elevator and the side wall portion. Therefore, cleaning using a tool such as a brush can be performed easily.

An endoscope according to a seventh aspect is so constituted that, in one of the first to fourth aspects, the distal end portion body has a pair of side wall portions facing each other having the elevator housing slit therebetween, one side wall portion of the pair of side wall portions has a notch portion which is formed by notching a corner portion on the side of the front surface, the corner portion being on the side of the lower surface, the notch portion is configured to allow the side surface of the elevator to be exposed when the elevator is displaced to the reclining position, and another side wall portion of the pair of side wall portions has a first thick portion which is provided on the side of the upper surface rather than the lower surface and has a first thickness, and a second thick portion which is provided between the first thick portion and the lower surface and has a second thickness smaller than the first thickness. In the seventh aspect, the side surface of the elevator is exposed by the notch portion, and moreover, since a gap corresponding to the difference between the first thickness and the second thickness is generated between the elevator and the side wall portion, the distal end portion can be cleaned more rapidly and easily.

An endoscope according to an eighth aspect is so constituted that, in one of the first to fourth aspects, the distal end portion body has a pair of side wall portions facing each other having the elevator housing slit therebetween, and the elevator has a recess portion on a surface facing any one side wall portion of the pair of side wall portions. In the eighth aspect, since the elevator has the recess portion, a gap is generated by this recess portion between the elevator and the side wall portion, cleaning using a tool such as a brush can be performed more rapidly and easily.

An endoscope according to a ninth aspect is so constituted that, in one of the first to eighth aspects, when a position where the elevator abuts on the partition wall portion of the cap is assumed to be a first reclining position in a state in which the cap is attached to the distal end portion body, and a position where the elevator is displaced from the first reclining position toward a side opposite to the erecting position is assumed to be a second reclining position in a state in which the cap is removed from the distal end portion body, the operating member displaces the elevator to the first reclining position when a first operation is performed and displaces the elevator to the second reclining position when a second operation is performed.

In the ninth aspect, the operation which displaces the elevator to the first and second reclining positions can be performed by the first and second operations of the operating member, respectively. When the elevator is at the second reclining position, it is exposed from the distal end portion body more largely than at the first reclining position, and thus, cleaning of the distal end portion can be performed more rapidly and easily.

An endoscope according to a tenth aspect is so constituted that, in the ninth aspect, the operation portion has a locking portion provided on the operating member and a stopper which is configured to restrict one end of a rotation range of the elevator to the first reclining position by engaging with the locking portion when the first operation is performed, and when the second operation is performed, the locking portion rides over the stopper and the engagement between the locking portion and the stopper is unlocked so as to allow movement of the elevator toward the second reclining position. In the tenth aspect, when the first operation is performed, the one end of the rotation range of the elevator is restricted to the first reclining position by the locking portion and the stopper, and thus, unintended reclining of the elevator can be prevented. When the elevator is to be further reclined beyond the first reclining position, by performing the second operation, the elevator is reclined to the second reclining position and is largely exposed from the distal end portion body. As a result, in the tenth aspect, cleaning of the distal end portion can be performed more rapidly and easily.

As described above, according to the endoscope of the present invention, the distal end portion can be cleaned rapidly and easily.

### BRIEF DESCRIPTION OF THE DRAWINGS

FIG. 1 is a view illustrating an entire configuration of an endoscope according to a first embodiment;

FIG. 2 is a perspective view illustrating a configuration of a distal end portion of the endoscope according to the first embodiment;

FIGS. 3A and 3B are views illustrating a distal end portion of the endoscope according to the first embodiment, in which FIG. 3A is a view illustrating a state when seen from a distal end direction, and FIG. 3B is a view illustrating a state when seen from above;

FIG. 4 is a view illustrating a state in which an elevator erecting lever and an elevator are connected in the endoscope according to the first embodiment;

FIGS. 5A and 5B are views illustrating a shape and a dimension of the elevator in the endoscope according to the first embodiment;

FIG. 6 is a view illustrating a shape and a dimension of an elevator housing slit in the endoscope according to the first embodiment;

FIG. 7 is a view illustrating a state in which the elevator is housed in the elevator housing slit in the endoscope according to the first embodiment;

FIG. 8 is a view illustrating a state in which the distal end portion of the endoscope according to the first embodiment is seen from a side surface direction (elevator erecting lever side);

FIG. 9 is another view illustrating a state in which the distal end portion of the endoscope according to the first embodiment is seen from the side surface direction;

FIG. 10 is still another view illustrating a state in which the distal end portion of the endoscope according to the first embodiment is seen from the side surface direction;

FIG. 11 is a conceptual diagram illustrating a stopper mechanism of the elevator provided on an operation portion of the endoscope according to the first embodiment;

FIG. 12 is a perspective view illustrating a distal end portion of an endoscope according to a second embodiment;

FIG. 13 is another view illustrating a state in which the distal end portion of the endoscope according to the second embodiment is seen from a side surface direction;

FIG. 14 is another view illustrating a state in which the distal end portion of the endoscope according to the second embodiment is seen from the side surface direction;

FIG. 15 is a view illustrating a state in which the distal end portion of the endoscope according to the second embodiment is seen from below;

FIG. 16 is a view illustrating a state in which the distal end portion of the endoscope according to the second embodiment is seen from a distal end side; and

FIGS. 17A and 17B are views illustrating a variation of the elevator.

### DETAILED DESCRIPTION OF THE PREFERRED EMBODIMENTS

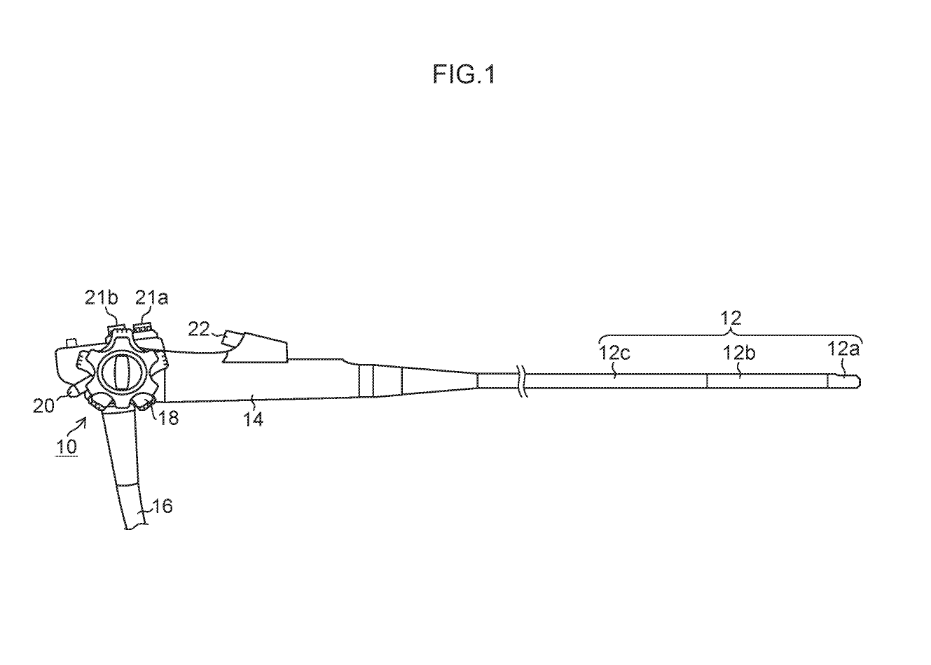

An endoscope according to the present invention will be described below by referring to the attached drawings. FIG. 1 is a view illustrating an entire configuration of the endoscope 10 according to the first embodiment.

### First Embodiment

### Entire Configuration of Endoscope

As illustrated in FIG. 1, the endoscope 10 includes an insertion portion 12 to be inserted into the body of a subject, and an operation portion 14 is coupled with a proximal end side of the insertion portion 12. To the operation portion 14, a universal cord 16 is connected, and the endoscope 10 is connected to a light source device, not shown, an image processing device (processor) and the like through this universal cord 16.

### <Entire Configuration of Insertion Portion>

The insertion portion 12 is constituted by sequentially connecting a distal end portion 12a, a bending portion 12b, and a flexible portion 12c from the distal end side toward the proximal end side (operation portion 14 side). On a farthest proximal end side of the flexible portion 12c, a boot (boot member) for the insertion portion 12 is provided.

### <Configuration of Operation Portion>

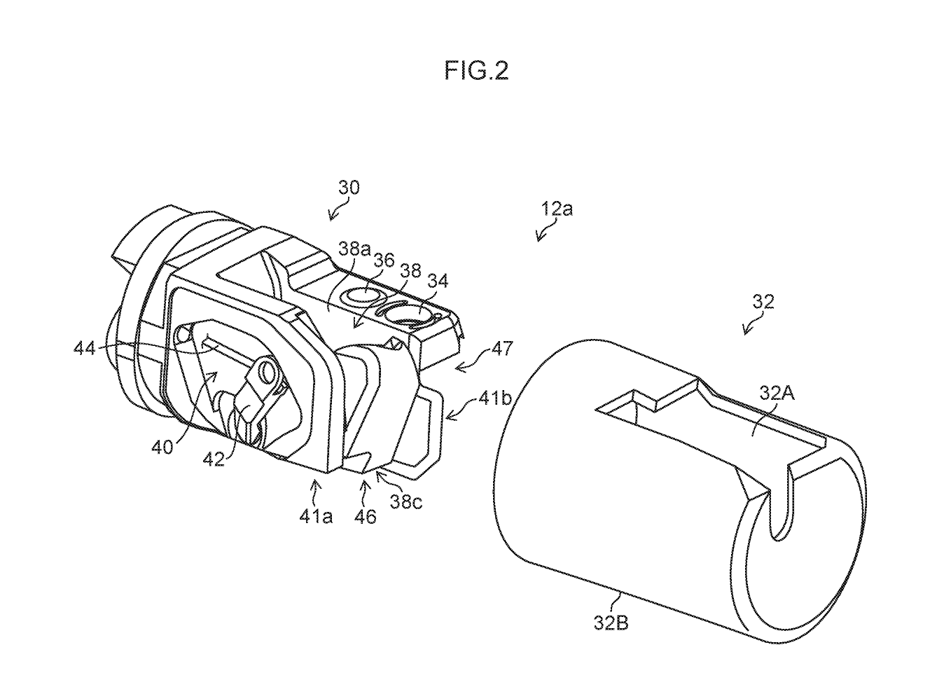

In the operation portion 14, an angle knob 18 which performs a bending operation of the bending portion 12b, an operation lever 20 which displaces (erects or reclines) the elevator 46 (see FIGS. 2, 4, and 5), an air/water supply button 21a which jets air, water and the like from an air/water supply nozzle provided on the distal end of the insertion portion 12, an suction button 21b and the like are provided. The operation lever 20 is an example of the operating member which operates the elevator. On the insertion portion 12 side of the operation portion 14, a treatment-tool entry port 22 into which various treatment tools such as forceps is inserted, is provided.

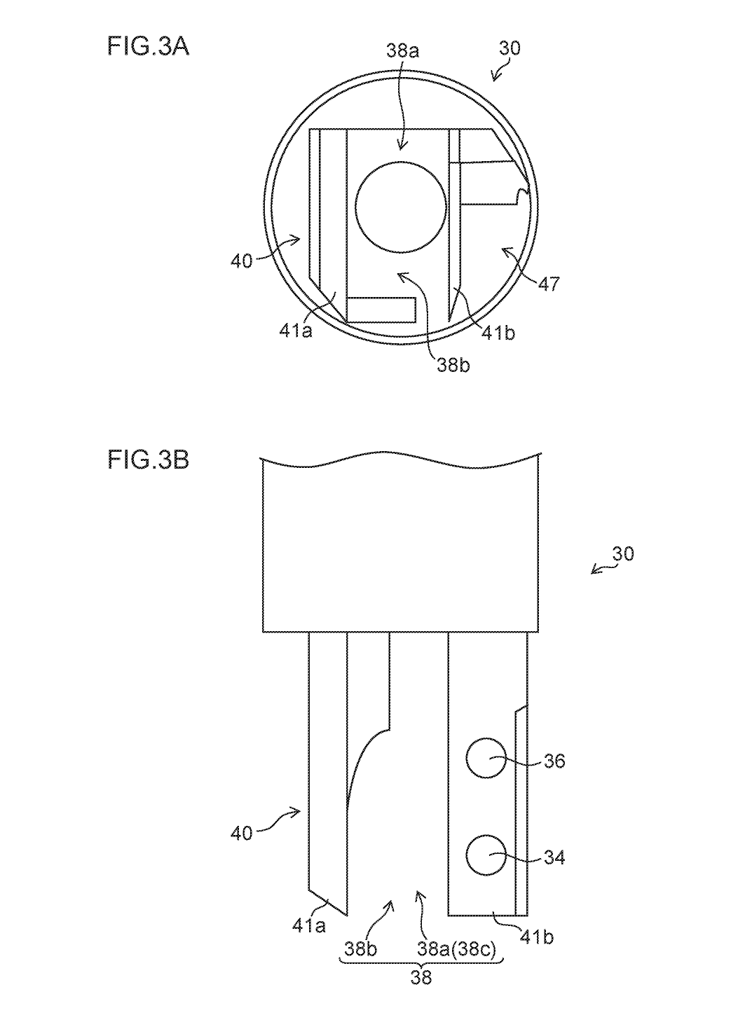

Moreover, in accordance with the operation of the air/water supply button 21a, air or water is supplied from an air/water supplying device built in the light source device, not shown, and is injected from the air/water supply nozzle toward an observation window 36 (see FIGS. 2 and 3). On the other hand, in accordance with the operation of the suction button 21b, suction is performed through an opening, not shown, provided on the distal end portion 12a. The distal end of the treatment tool inserted into the treatment-tool entry port 22 is led out from the opening of the distal end portion 12a through a forceps channel, not shown, communicating with the treatment-tool entry port 22, and a direction of the treatment tool can be changed by displacing (erecting or reclining) the elevator 46 by operating the operation lever 20.

### <Configuration of Flexible Portion>

The flexible portion 12c has a configuration in which: the innermost side is a spiral tube formed by spirally braiding a thin band-shaped plate with elasticity on the innermost side, the spiral tube is then covered with a net-like body that is woven from a metal wire and fitted with a metal cap at both ends thereof to form a tubular body; the outer peripheral surface of the tubular body is laminated with an outer skin formed of resin.

### <Configuration of Bending Portion>

The bending portion 12b has a configuration in which: a structural body is constituted by rotatably coupling angle rings, not shown, with each other; an outer periphery of this structural body is covered by a mesh body obtained by braiding a metal wire; and the mesh body is further covered by an outer skin made of rubber. Moreover, a plurality of wires, not shown, are extended from the angle knob 18 of the operation portion 14 to the bending portion 12b, and distal end portions of these wires are fixed to the distal end portions of the angle rings constituting the bending portion 12b. As a result, in accordance with the operation of the angle knob 18, the bending portion 12b is bent vertically and horizontally.

### <Configuration of Distal End Portion>

FIG. 2 is an exploded perspective view illustrating a structure of the distal end portion 12a according to this embodiment. As illustrated in FIG. 2, the distal end portion 12a has a cap 32 covering a distal end portion body (body of the distal end portion) 30. In the cap 32, an opening window 32A which opens an opening portion 38a on the upper surface side of an elevator housing slit 38 (which will be described later) and a partition wall portion 32B which closes an opening portion 38c on the lower surface side are formed, in a state where the cap 32 is attached to the distal end portion body 30. In the state where the cap 32 is attached, reclining of the elevator 46 is regulated by the partition wall portion 32B. Moreover, the cap 32 is made of a material with an elastic force or silicone rubber, for example, and by fitting a small diameter portion with a small inner diameter provided on a proximal end of the cap 32 into a groove formed in the distal end portion body 30, the cap 32 is detachably attached to the distal end portion body 30.

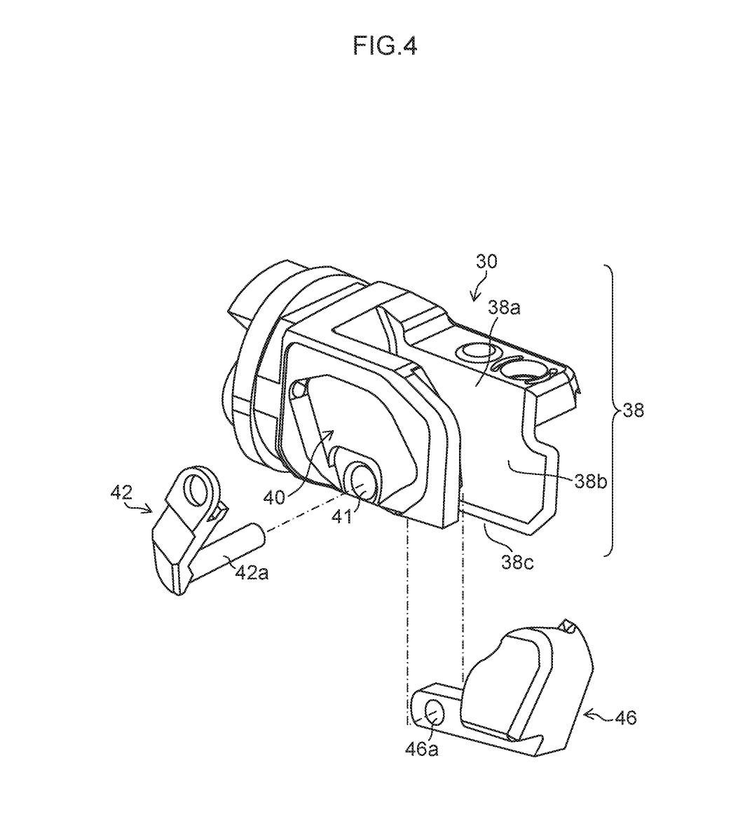

The distal end portion body 30 is constituted by a metal with good erosion resistance such as stainless steel, and the elevator housing slit 38 (see FIGS. 2 to 4) is provided at a center position of the distal end portion body 30. This elevator housing slit 38 forms a space portion which houses the elevator 46. As illustrated in FIGS. 3A, 3B and 4, opening portions 38a, 38b, and 38c are opened and extends from the upper surface of the distal end portion body 30 (on the forceps (treatment tool) exit port side from the rotating shaft 42a as a starting point) to the lower surface (on a side opposite to the forceps (treatment tool) derivation direction from the rotating shaft 42a as the starting point) through the front surface (distal end side of the distal end portion body 30). Moreover, the distal end portion body 30 has a pair of side wall portions 41a and 41b faced with each other having the elevator housing slit 38 between them.

In FIGS. 2 to 4, in order to explicitly illustrate the opening portions 38a, 38b, and 38c, illustration of the cap 32, the elevator erecting lever 42, the elevator 46 and the like is omitted as appropriate.

As described above, in the endoscope 10 according to this embodiment, since the opening portions 38a, 38b, and 38c extending from the upper surface to the lower surface through the front surface are formed in the elevator housing slit 38, the exposed range of the elevator 46 is large, and the elevator 46 can be cleaned rapidly and easily.

Moreover, a treatment-tool insertion channel, not shown, communicates with the elevator housing slit 38. This treatment-tool insertion channel is inserted into the insertion portion 12 of the endoscope 10 and is connected to the treatment-tool entry port 22 of the operation portion 14. As a result, when the treatment tool is inserted into the treatment-tool insertion channel from the treatment-tool entry port 22, the treatment tool is guided to the elevator housing slit 38. The guided treatment tool is erected by the elevator 46 and is led out upward from the opening window 32A.

In one side wall portion 41a of the elevator housing slit 38, on a surface on a side opposite to the elevator 46, an erecting-lever housing chamber 40 is provided, and inside this erecting-lever housing chamber 40, an elevator erecting lever 42 which causes the elevator 46 to swing is housed. Moreover, on the side wall portion 41b on the side opposite to the erecting-lever housing chamber 40 having the elevator housing slit 38 between them, an optical-system housing chamber 47 is provided.

The optical-system housing chamber 47 is closed by attaching a protective cover, not shown, to the distal end portion body 30. A joint surface between the distal end portion body 30 and the protective cover is joined through a sealing agent, whereby a gap between the distal end portion body 30 and the protective cover is sealed, and airtightness of the optical-system housing chamber 47 is held.

On the upper part of the optical-system housing chamber 47, an illumination window 34 and the observation window 36 are disposed, and an air/water supply port, not shown, is provided toward the observation window 36. The air/water supply port is connected to an external device through an air/water supply tube (not shown) inserted into the insertion portion 12. Compressed air or water is injected from the air/water supply port toward the observation window 36 by operating the air/water supply button 21a of the operation portion 14, and the observation window 36 is cleaned. The illumination window 34, the observation window 36, and the air/water supply port are provided at a position facing the opening window 32A when the cap 32 is placed over the distal end portion body 30. Hereinafter, a surface on which the observation window 36 is disposed is referred to as an upper surface, and an observation direction observed from the observation window 36 is referred to as an upward. Moreover, the surface on a side opposite to the upper surface or upward is referred to as a lower surface or downward, and the surface on the distal end side of the distal end portion 12a is referred to as a front surface or forward as a front part.

Inside the optical-system housing chamber 47, an imaging optical system and an illumination optical system, not shown, are housed. The illumination optical system includes an illumination lens (not shown) which is installed on an inner side of the illumination window 34 and a light guide which is arranged so that a distal end the light guide faces this illumination lens. The light guide is inserted into the insertion portion 12 of the endoscope 10 and the proximal end portion thereof is connected to the light source device, not shown. As a result, an illumination light from the light source device is transmitted through the light guide and is projected upward from the illumination window 34.

The imaging optical system includes an objective lens disposed on an inner side of the observation window 36 and a CCD unit (Charge Coupled Device unit), not shown, disposed in the rear of this objective lens through a prism. The CCD unit is connected to an image processing device, not shown, through a cable. By means of this imaging optical system, a subject image taken in from the observation window 36 is converted to an electric signal and transmitted to the image processing device (processor) connected to the endoscope 10 and the subject image is displayed on a monitor through the image processing device.

On the other hand, the erecting-lever housing chamber 40 is closed by placing a protective plate, not shown, over the distal end portion body 30. The protective plate is screwed to the distal end portion body 30 at a plurality of spots (not shown), and a gap between the protective plate and the distal end portion body 30 is filled with a sealing agent. As a result, airtightness of the erecting-lever housing chamber 40 is maintained.

### <Shape and Dimension of Elevator>

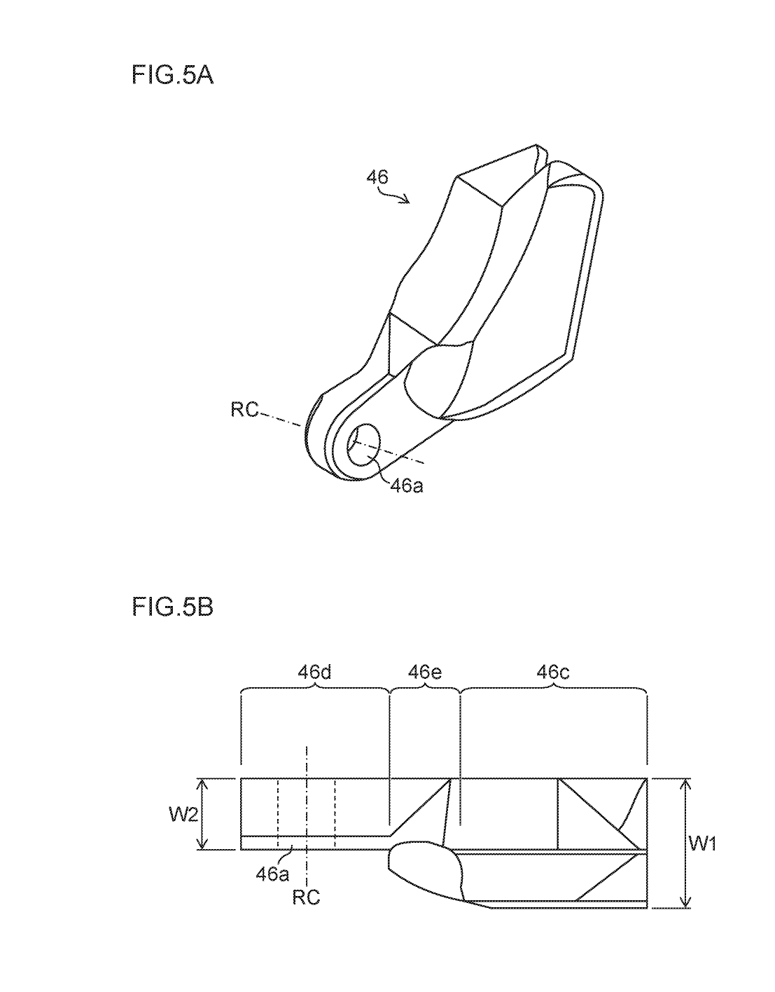

FIGS. 5A and 5B are views illustrating the elevator 46 in this embodiment. FIG. 5A is a perspective view of the elevator 46, and FIG. 5B is a view illustrating the elevator 46 from an upper surface direction. As illustrated in FIG. 5B, the elevator 46 has: a forceps action region 46c which has a first width W1 and receives forceps; a rotation center region 46d which has a second width W2 narrower than the first width W1 and includes a rotation center RC (virtual center axis of rotation) of the elevator 46; and an elevator width transition region 46e which is located between the forceps action region 46c and the rotation center region 46d and in which the width continuously changes within a range from W2 or more to W1 or less. In the forceps action region 46c, a center in the width direction is recessed, and this recess receives the forceps (various treatment tools) so as to lead it out to the upper surface side.

### <Shape and Dimension of Elevator Housing Slit>

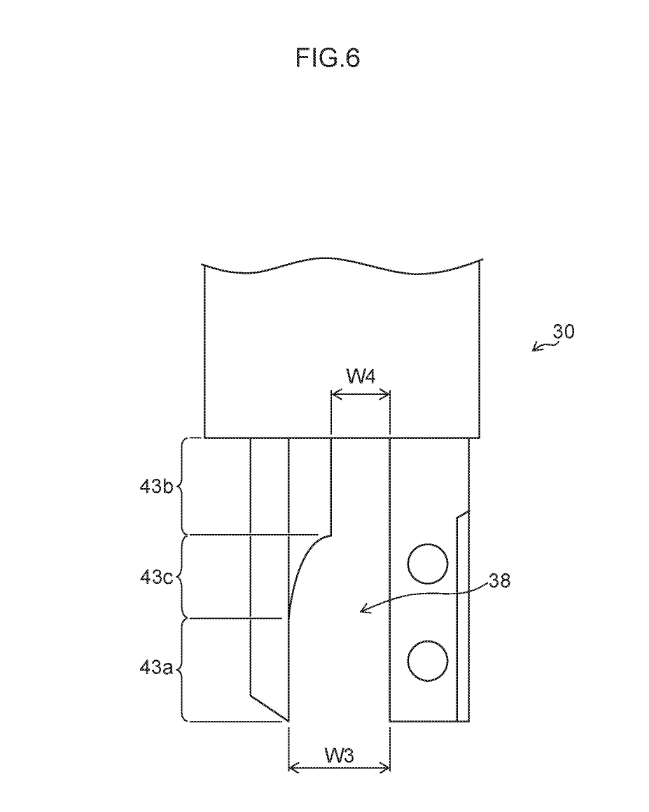

On the other hand, FIG. 6 is a view of the elevator housing slit 38 in this embodiment seen from the upper surface side. As illustrated in FIG. 6, the elevator housing slit 38 has: a distal-end-side region 43a which has a width W3 wider than the first width W1 and houses the forceps action region 46c; a proximal-end-side region 43b which has a fourth width W4 narrower than the third width W3 and wider than the second width W2 and houses the rotation center region 46d; a forceps housing slit width transition region 43c which is located between the distal-end-side region 43a and the proximal-end-side region 43b and in which the width continuously changes from the fourth width W4 to the third width W3.

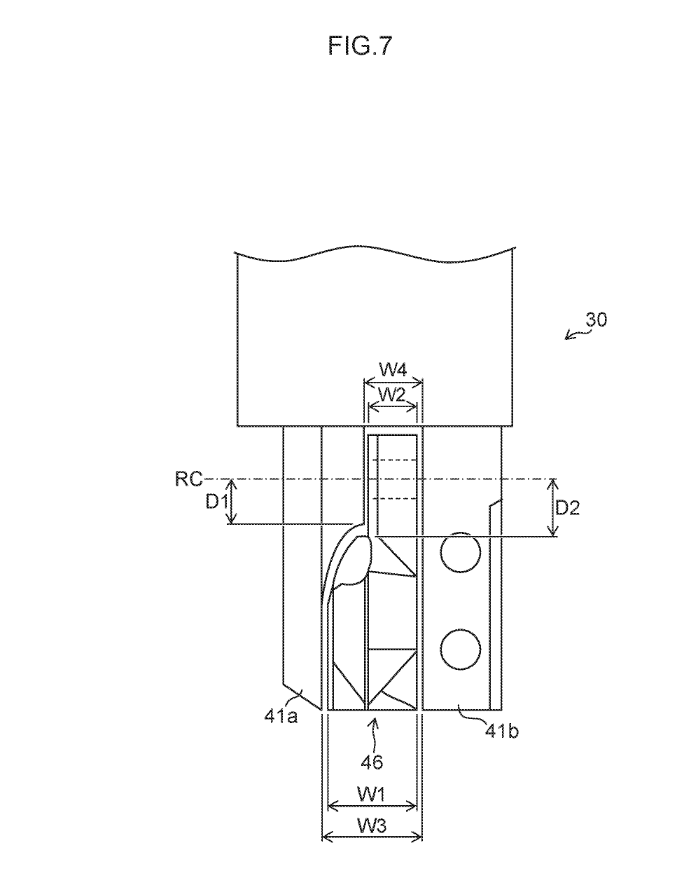

Therefore, in the endoscope 10 according to this embodiment, even if the elevator 46 is housed in the elevator housing slit 38, the elevator 46 and the elevator housing slit 38 are not brought into contact with each other as illustrated in FIG. 7, and the elevator 46 can be rotated around the rotation center RC.

### <Displacement of Elevator by Operation of Operating Member>

Inside the erecting-lever housing chamber 40, the elevator erecting lever 42 is provided (see FIGS. 2, 4, and 8 to 10). As illustrated in FIG. 4, the rotating shaft 42a provided on the lower end portion of the elevator erecting lever 42 is inserted into a hole 41 and is pivotally supported so as to be rotatable with respect to the distal end portion body 30, thereby constituting the rotating shaft of the elevator 46. Moreover, it is so constituted that a distal end of the rotating shaft 42a is fitted in a hole 46a of the elevator 46 so that the elevator erecting lever 42 is fixed to the elevator 46 so as not to be mutually unrotatable, and the elevator erecting lever 42 and the elevator 46 are integrally made to swing. Moreover, with the upper end portion of the elevator erecting lever 42, the distal-end-side coupling portion 44a of the operation wire 44 is coupled, and this operation wire 44 is inserted into the insertion portion 12, and the proximal-end-side coupling portion is coupled with the operation lever 20 of the operation portion 14. Therefore, by rotating operation of the operation lever 20, the operation wire 44 is pushed and pulled so that the elevator erecting lever 42 is rotated, and as a result, the elevator 46 is displaced (erect or recline). The elevator erecting lever 42 is a member constituting the elevator erecting mechanism in the present invention. Moreover, the shaft center of the rotating shaft 42a matches the rotation center RC.

In this embodiment, as the elevator erecting mechanism, the case in which the elevator erecting lever 42 is provided in order to rotate the elevator 46, and the elevator 46 and the rotating shaft 42a are fixed is described, but the elevator erecting mechanism in the present invention is not limited to this embodiment. In addition to the embodiment above, the elevator erecting mechanism may be so constituted that a shaft-shaped member constituting the rotating shaft is fixed to the distal end portion body and the elevator is rotated around the fixed member directly by the operation wire without through the elevator erecting lever (similarly in the second embodiment which will be described later).

### <Displacement of Elevator: Normal Use>

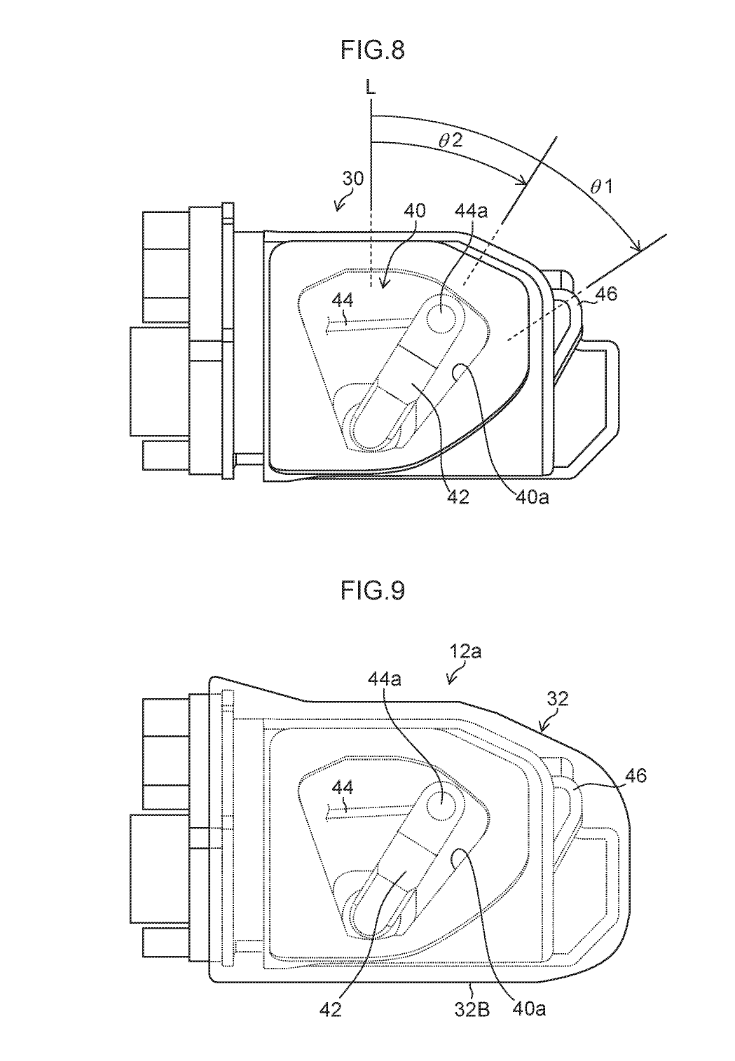

FIG. 8 is a view illustrating the distal end portion 12a in normal use of the endoscope 10. FIG. 8 illustrates a state in which the elevator 46 is at a first reclining position (a position where the elevator 46 is reclined toward the distal end side and abuts on the partition wall portion 32B of the cap 32). The endoscope 10 is used in a state with the protective plate placed over the erecting-lever housing chamber 40 as described above, but in FIG. 8, in order to explicitly illustrate the reclining state of the elevator erecting lever 42, illustration of the protective plate is omitted, and the erecting-lever housing chamber 40, the elevator erecting lever 42 and the like covered by the protective plate are indicated by a dotted line (the same applies to FIGS. 9, 10, 13, and 14). In FIG. 8, illustration of the cap 32 is omitted, and in FIG. 9, the cap 32 is illustrated. As described above, on the elevator housing slit 38, the opening portions 38a, 38b, and 38c extending from the upper surface to the lower surface through the front surface are formed. Since the reclining of the elevator 46 is regulated by the partition wall portion 32B in the normal use and moreover, since a stopper mechanism 60 (which will be described later) is provided on the operation portion 14, the elevator 46 is prevented from reclining toward the lower surface side from the state illustrated in FIGS. 8 and 9.

### <Displacement of Elevator: Cleaning>

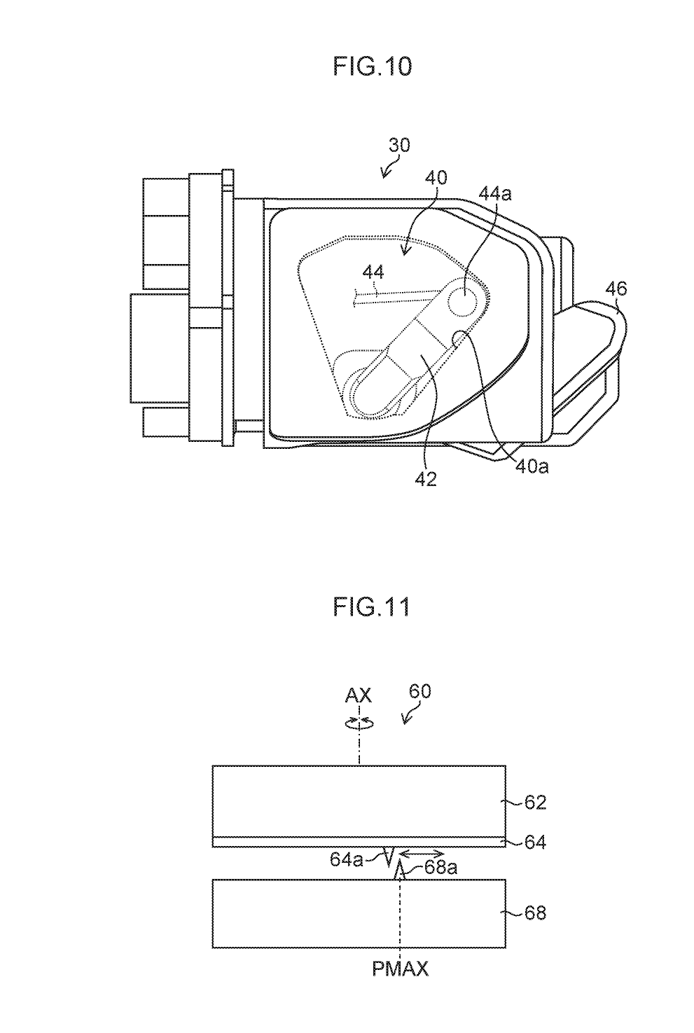

On the other hand, FIG. 10 is a view illustrating a state of the distal end portion 12a in cleaning. FIG. 10 illustrates a state in which the cap 32 is removed and moreover, the elevator 46 is further reclined toward the lower surface side from the first reclining position (state in FIGS. 8 and 9) to a second reclining position by a second operation (which will be described later) of the operation lever 20. In the state in FIG. 10, exposure (exposure of the side surface portion) of the elevator 46 from the elevator housing slit 38 increases compared to the state in FIGS. 8 and 9 and thus, a range that can be cleaned easily increases.

### <Elevator Erecting Lever and Reclining Angle of Elevator>

As illustrated in FIG. 8, in the endoscope 10 according to this embodiment, a reclining angle θ1 of the elevator 46 is larger than a reclining angle θ2 (an angle based on a direction L orthogonal to the axial direction of the endoscope 10) of the elevator erecting lever 42 (that is, θ1>θ2). That is, when the elevator 46 is erected at the first reclining position, the elevator erecting lever 42 is inclined (erecting) to the proximal end side of the distal end portion 12a larger than the elevator 46 with respect to the rotating shaft 42a (the shaft center matches the rotation center RC of the elevator 46). Therefore, when the erecting state is to be cancelled, that is, when the elevator 46 is to be reclined to the second reclining state (see FIG. 10) by the second operation of the operation lever 20, before the elevator erecting lever 42 abuts on an end portion 40a (see FIGS. 8 to 10) of the erecting-lever housing chamber 40 so as to restrict further reclining of the elevator erecting lever 42 and the elevator 46 (before the state shown in FIG. 10), the side surface of the elevator 46 is further exposed from the opening portion 38b on the front surface side or the opening portion 38c on the lower surface side of the elevator housing slit 38. In the endoscope 10 according to this embodiment, the elevator 46 and the distal end portion 12a can be cleaned rapidly and easily due to such exposure.

Moreover, assuming that the reclining angle satisfies θ1≦θ2, in order to largely recline the elevator 46 (to the second reclining position illustrated in FIG. 10, for example), the elevator erecting lever 42 also needs to be largely reclined. Therefore, the erecting-lever housing chamber 40 should be made large in order to ensure a rotation space of the elevator erecting lever 42. However, in the endoscope 10 according to this embodiment, by setting the reclining angles θ1 and θ2 to have the relation described above, the reclining angle θ1 of the elevator 46 can be made larger than the reclining angle θ2 of the elevator erecting lever 42, and the rotation space of the elevator erecting lever 42 is reduced, and the erecting-lever housing chamber 40 and the distal end portion body 30 including it and the distal end portion 12a can be downsized.

### <Stopper Mechanism>

Subsequently, a stopper mechanism (locking mechanism) of the elevator will be described. FIG. 11 is a conceptual diagram illustrating a structure of the stopper mechanism 60. As illustrated in FIG. 11, in the endoscope 10, a movable portion 62 which is rotatable around an axis AX in conjunction with (in interlock with) the rotating operation of the operation lever 20 is provided in the operation portion 14, and to a lower surface of this movable portion 62, a plate spring 64 including a projection 64a is attached. On the other hand, on a fixed portion 68 provided in a fixed manner on the body side of the operation portion 14, a stopper 68a is provided on a surface facing the movable portion 62. The projection 64a and the plate spring 64 are members constituting a locking portion in the present invention.

The stopper 68a is provided at a position PMAX corresponding to the first reclining position of the elevator 46. This stopper 68a is an example of a stopper in the present invention. Therefore, by rotating the operation lever 20 so as to allow the projection 64a to engage with the stopper 68a, one end of the rotation range of the elevator 46 is restricted (locked) to the first reclining position. Such operation of the operation lever 20 is referred to as the “first operation” hereinafter.

Therefore, in order to make the elevator 46 further recline over the first reclining position, an operation different from the rotating operation in the normal use of the operation lever 20 (an operation to intentionally apply a force larger than the normal use to further recline the elevator 46: hereinafter referred to as the “second operation”) needs to be performed to the operation lever 20, and thus, the reclining from the first reclining position toward the second reclining position (position further displaced to a side opposite to the erecting position from the first reclining position; the state illustrated in FIG. 10) is restricted, and unintended reclining of the elevator 46 and resulting breakage of the cap 32 can be prevented. Moreover, when the distal end portion 12a is to be cleaned, by performing the second operation, the projection 64a rides over the stopper 68a, and engagement between the projection 64a and stopper 68a is unlocked, whereby the elevator 46 is reclined to the second reclining position and is largely exposed from the elevator housing slit 38 (see FIG. 10). In this state, the distal end portion 12a can be cleaned more rapidly and easily.

In the stopper mechanism 60, the number and size of the projection 64a or the stopper 68a may be determined in accordance with intensity of a required locking force.

### Second Embodiment

Subsequently, a second embodiment of an endoscope according to the present invention will be described. In the second embodiment, the configuration similar to that in the first embodiment is given the same reference numeral and detailed description will be omitted.

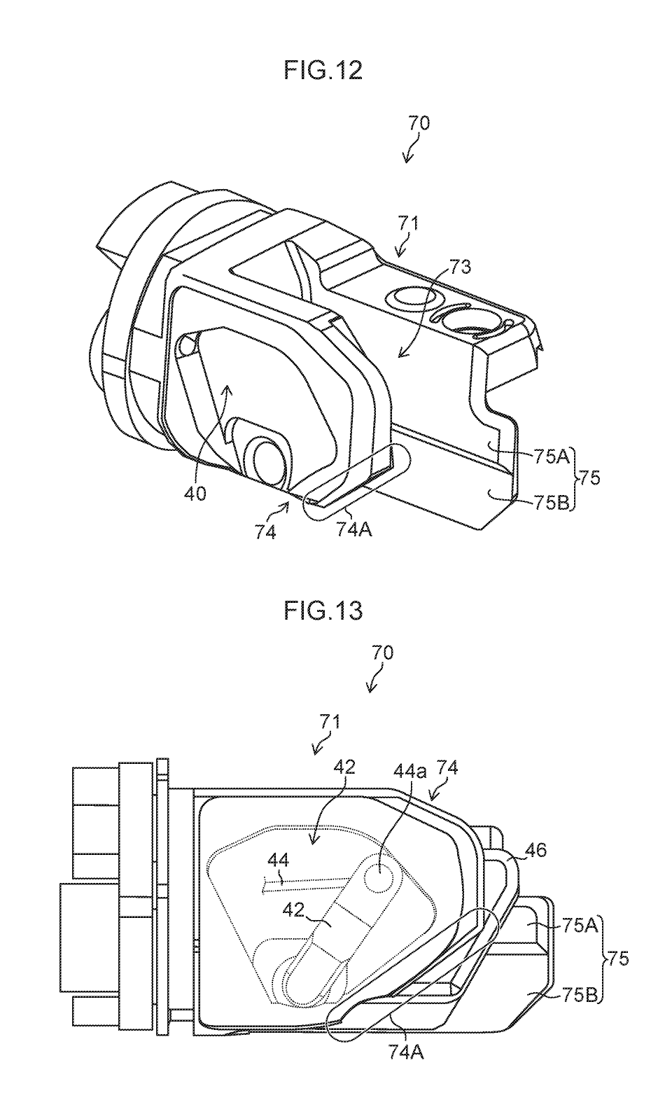

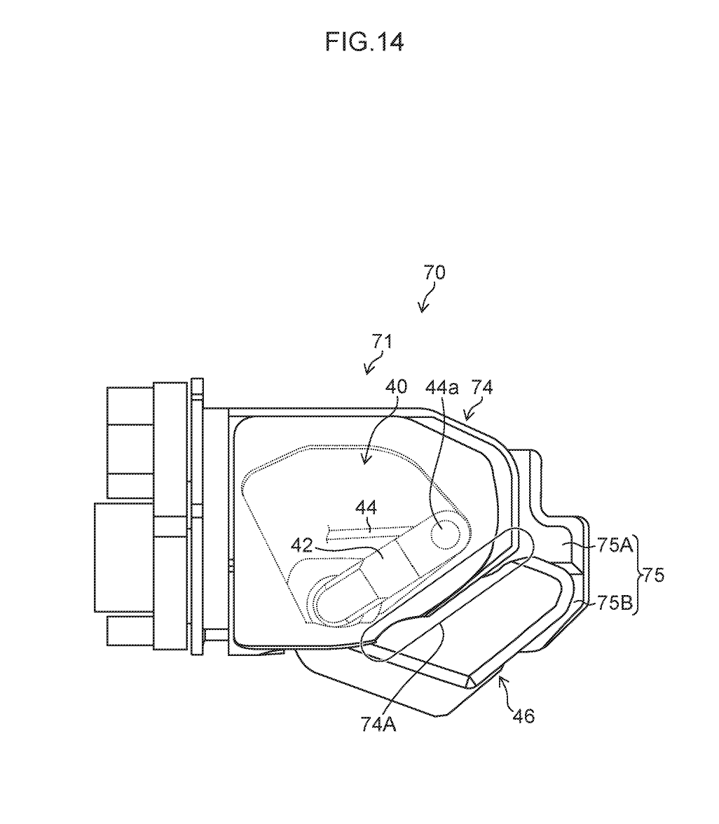

Basic configuration of the endoscope according to the second embodiment is similar to that of the endoscope 10 according to the first embodiment, but in the second embodiment, a structure of the distal end portion is different from that in the first embodiment. FIG. 12 is a view illustrating a distal end portion body 71 according to the second embodiment (illustration of the cap 32, the elevator 46, and the elevator erecting lever 42 is omitted), FIG. 13 relates to the second embodiment and is a view illustrating a distal end portion 70 with the elevator 46 at the first reclining position (illustration of the cap 32 is omitted; the same applies to FIG. 14), and FIG. 14 is a view illustrating the distal end portion 70 in cleaning (a state in which the cap 32 is removed, and the elevator 46 is displaced to the second reclining position). In the second embodiment, also, similarly to the first embodiment, the elevator 46 can be displaced by the first operation and second operation of the operation lever 20 to the first reclining position and second reclining position, respectively. Moreover, in the second embodiment, also, similarly to the first embodiment, the stopper mechanism 60 is provided.

As illustrated in FIGS. 12 to 14, the distal end portion body 71 has a pair of side wall portions 74 and 75 facing each other having an elevator housing slit 73 therebetween. The side wall portion 74 (side wall portion on the erecting-lever housing chamber 40 side) has a notch portion 74A on the front surface side, the notch portion 74A which is formed by notching (cutting out) a corner portion on the lower surface side from its bottom to upward (indicated by a circle in FIGS. 12 to 14). Thus, as illustrated in FIGS. 13 and 14, the side surface of the elevator 46 (side surface on the erecting-lever housing chamber 40 side) is largely exposed from the elevator housing slit 73 and the distal end portion 70 can be cleaned rapidly and easily. In the distal end portion 70 in the second embodiment, also, similarly to the distal end portion 12a according to the first embodiment, the elevator housing slit 73 is penetrated from the upper surface to the bottom surface so as to be opened.

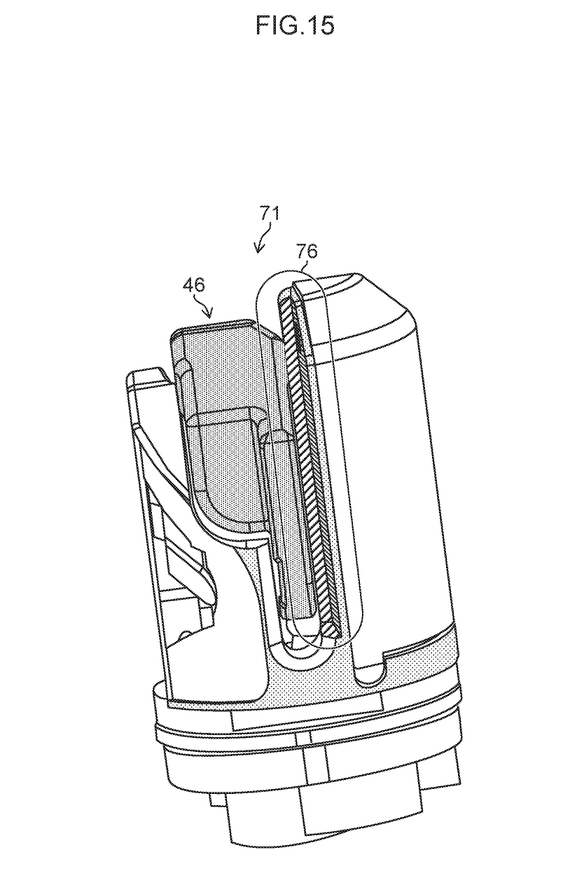

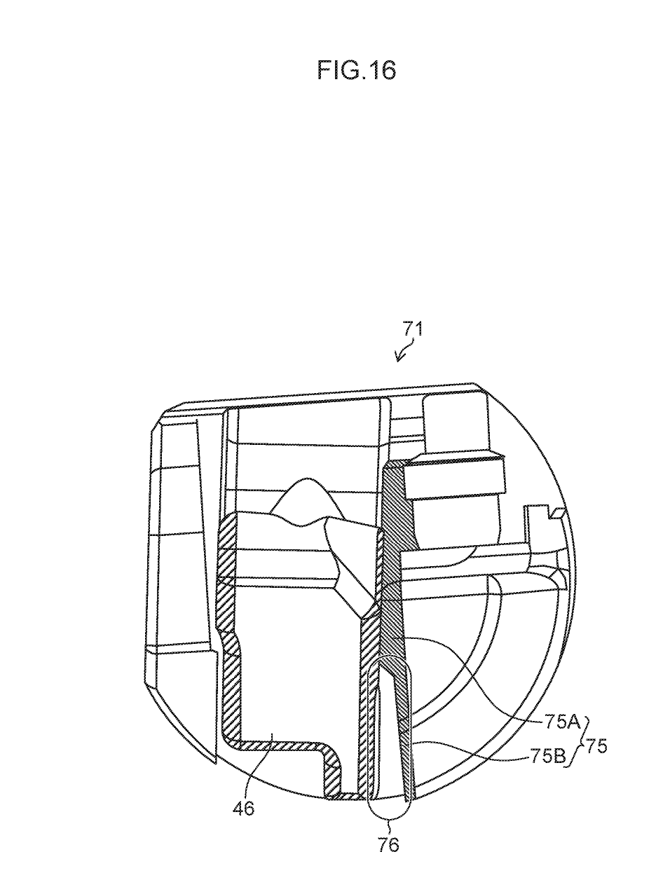

On the other hand, in the side wall portion 75 (side wall portion on the optical-system housing chamber 47 side), the upper surface side is a first thick portion 75A having a first thickness and a portion between the first thick portion 75A and the lower surface is a second thick portion 75B having a second thickness smaller than the first thickness. As a result, as illustrated in FIG. 15 (state in which the distal end portion 70 is seen from below) and FIG. 16 (state in which the distal end portion 70 is seen from the distal end side), a gap 76 (indicated by a circle in FIGS. 15 and 16) is formed between the elevator 46 and the side wall portion 75 (second thick portion 75B), into which bristles of a brush and the like can easily enter, and the distal end portion 70 can be cleaned rapidly and easily. In FIGS. 15 and 16, some parts of members are shaded in order to explicitly illustrate the gap 76.

In the second embodiment, also, similarly to the first embodiment (see FIG. 8), by making the reclining angle θ1 of the elevator 46 larger than the reclining angle θ2 of the elevator erecting lever 42 (that is, θ1>θ2), the distal end portion body 71 and the distal end portion 70 are downsized, and rapid and easy cleaning is made possible.

In the second embodiment, the case in which the notch portion 74A is provided on the side wall portion 74 on the erecting-lever housing chamber 40 side, and the first thick portion 75A and the second thick portion 75B are provided on the side wall portion 75 on the optical-system housing chamber 47 side is described, but the configuration of the notch portion and the first and second thick portions in the present invention is not limited to this embodiment. Only the notch portion or one of the first and second thick portions may be provided on one of the side wall portions. Or, to the contrary to the second embodiment, the first and second thick portions may be provided on the side wall portion 74, while the notch portion is provided on the side wall portion 75.

Moreover, in the second embodiment, also, the shapes and the dimensions of the elevator 46 and the elevator housing slit 38 are in the relationship similar to that of the first embodiment (see FIGS. 5A to 7).

### <Variation of Elevator>

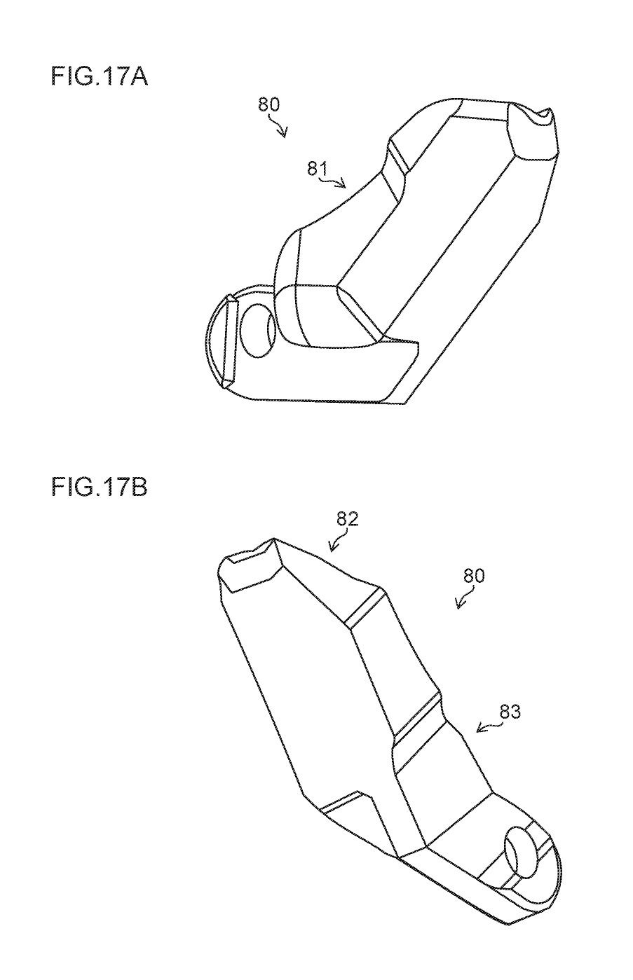

Subsequently, a variation of the elevator in the present invention will be described. In the aforementioned second embodiment, by providing the notch portion or thin portion (second thick portion) on the side wall portions 74 and 75, a gap is formed between the elevator 46 and the side wall portions 74 and 75, whereby cleaning is facilitated. However, the shape of the elevator may be changed for forming the gap. FIGS. 17A and 17B are views illustrating an elevator 80 formed in such a shape, in which FIG. 17A illustrates a state seen from the elevator erecting lever 42 side, and FIG. 17B illustrates a state when seen from the optical-system housing chamber 47 side. As illustrated in FIGS. 17A and 17B, in the elevator 80, a narrow portion 81 is formed on the side surface portion on the elevator erecting lever 42 side, and notch portions 82 and 83 are formed on the side surface on the optical-system housing chamber 47. Therefore, the gap is formed between the elevator 80 and the side wall portion when the elevator 80 is housed in the elevator housing slit, and cleaning by a tool such as a brush and the like can be performed rapidly and easily.

The narrow portion 81 and the notch portions 82 and 83 are examples of a recess portion (concave portion) of the elevator in the present invention. Moreover, the shape of the elevator illustrated in this variation may be employed in either of the first and second embodiments.

The present invention is not limited to the aforementioned embodiments but is capable of various variations within a range not departing from the spirit of each invention.

What is claimed is:

1. An endoscope, comprising:
an insertion portion which includes a distal end and a proximal end;
an operation portion which is provided on a proximal end side of the insertion portion and includes an operating member;
a distal end portion body which is provided on a distal end side of the insertion portion, and has a front surface, an upper surface, and a lower surface;
an elevator which has a rotation center and is configured to be rotatable with respect to the distal end portion body;
an elevator erecting mechanism configured to rotate the elevator around the rotation center;
an operation wire which includes a proximal-end-side coupling portion coupled with the operating member and a distal-end-side coupling portion coupled with the elevator erecting mechanism, the operation wire configured to rotate the elevator by being pushed or pulled by an operation of the operating member, and displace the elevator between an erecting position and a reclining position;
an elevator housing slit which is provided on the distal end portion body and forms a space portion housing the elevator, the elevator housing slit being extended and opened from the upper surface to the lower surface through the front surface of the distal end portion body; and
a cap detachably provided on the distal end portion body, the cap including an opening window which opens the opening portion on a side of the upper surface of the elevator housing slit and a partition wall portion which closes the opening portion on a side of the lower surface in a state in which the cap is attached to the distal end portion body.

2. The endoscope according to claim 1, wherein
the elevator has:
a forceps action region which has a first width and is configured to receive forceps;
a rotation center region which has a second width narrower than the first width and includes the rotation center of the elevator; and
an elevator width transition region which is disposed between the forceps action region and the rotation center region, and whose width changes from the second width to the first width, and
the elevator housing slit has:
a distal-end-side region which has a third width wider than the first width and is configured to house the forceps action region;
a proximal-end-side region which has a fourth width narrower than the third width and wider than the second width, and is configured to house the rotation center region, and
a forceps housing slit width transition region which is disposed between the distal-end-side region and the proximal-end-side region, and whose width changes from the fourth width to the third width, wherein
a distance from the rotation center to the forceps housing slit width transition region is shorter than a distance from the rotation center to the elevator width transition region.

3. The endoscope according to claim 1, wherein
the elevator erecting mechanism has an elevator erecting lever coupled with the elevator,
the distal-end-side coupling portion of the operation wire is coupled with the elevator erecting lever, and
the operation wire is pushed or pulled by an operation of the operating member so as to rotate the elevator through the elevator erecting lever and displace the elevator.

4. The endoscope according to claim 3, wherein
the elevator erecting lever is coupled with the elevator in an unrotatable manner, and
the elevator erecting lever is configured to be inclined larger than the elevator toward the proximal end side with respect to the rotation center when the elevator is erected, and
a side surface of the elevator is exposed from an opening portion on a side of the front surface of the elevator housing slit or the opening portion on the side of the lower surface of the elevator housing slit, before the elevator erecting lever abuts on the distal end portion body and further rotation of the elevator erecting lever and the elevator is restricted, when the erecting of the elevator is cancelled.

5. The endoscope according to claim 1, wherein
the distal end portion body has a pair of side wall portions facing each other having the elevator housing slit therebetween, and
at least one side wall portion of the pair of side wall portions has a notch portion which is formed by notching a corner portion on the side of the lower surface, the corner portion being on the side of the front surface, and
the notch portion is configured to allow the side surface of the elevator to be exposed when the elevator is displaced to the reclining position.

6. The endoscope according to claim 1, wherein
the distal end portion body has a pair of side wall portions facing each other having the elevator housing slit therebetween, and
at least one side wall portion of the pair of side wall portions has a first thick portion which is provided on the side of the upper surface rather than the lower surface and has a first thickness, and a second thick portion which is provided between the first thick portion and the lower surface and has a second thickness smaller than the first thickness.

7. The endoscope according to claim 1, wherein
the distal end portion body has a pair of side wall portions facing each other having the elevator housing slit therebetween,
one side wall portion of the pair of side wall portions has a notch portion which is formed by notching a corner portion on the side of the front surface, the corner portion being on the side of the lower surface,
the notch portion is configured to allow the side surface of the elevator to be exposed when the elevator is displaced to the reclining position, and
another side wall portion of the pair of side wall portions has a first thick portion which is provided on the side of the upper surface rather than the lower surface and has a first thickness, and a second thick portion which is provided between the first thick portion and the lower surface and has a second thickness smaller than the first thickness.

8. The endoscope according to claim 1, wherein
the distal end portion body has a pair of side wall portions facing each other having the elevator housing slit therebetween, and
the elevator has a recess portion on a surface facing any one side wall portion of the pair of side wall portions.

9. The endoscope according to claim 1, wherein
when a position where the elevator abuts on the partition wall portion of the cap is assumed to be a first reclining position in a state in which the cap is attached to the distal end portion body, and a position where the elevator is displaced from the first reclining position toward a side opposite to the erecting position is assumed to be a second reclining position in a state in which the cap is removed from the distal end portion body,
the operating member displaces the elevator to the first reclining position when a first operation is performed and displaces the elevator to the second reclining position when a second operation is performed.

10. The endoscope according to claim 9, wherein
the operation portion has a locking portion provided on the operating member and a stopper which is configured to restrict one end of a rotation range of the elevator to the first reclining position by engaging with the locking portion when the first operation is performed, and
when the second operation is performed, the locking portion rides over the stopper and the engagement between the locking portion and the stopper is unlocked so as to allow movement of the elevator toward the second reclining position.
